(19) United States
(12) Patent Application Publication
(10) Pub. No.: US 2024/0263146 A1
(43) Pub. Date: Aug. 8, 2024

(54) HUMAN NSCLC CELL LINES AND USES THEREOF

(71) Applicant: SHANGHAI LIDE BIOTECH CO., LTD., Shanghai (CN)

(72) Inventor: Danyi Wen, Shanghai (CN)

(21) Appl. No.: 18/008,443

(22) PCT Filed: Jun. 3, 2021
(86) PCT No.: PCT/CN2021/098081

§ 371 Date: Dec. 5, 2022

(30) Foreign Application Priority Data
Jun. 5, 2020 (WO) PCT/CN2020/094778

(51) Int. Cl. C12N 5/09; A01K 67/0275; C12N 5/071; G01N 33/50; G01N 33/574

(52) U.S. Cl. CPC C12N 5/0693; A01K 67/0275; C12N 5/0688; G01N 33/5011; G01N 33/5044; G01N 33/57423; A01K 2227/105; A01K 2267/0331

(57) ABSTRACT

The present invention relates to a human non-small cell lung cancer (NSCLC) cell line, and the use of the same in evaluating the efficacy of anti-NSCLC drugs. The present invention also discloses an animal model comprising the human NSCLC cell lines, method for producing the same and the use of the animal model in testing efficacy of drugs or obtaining pharmacological data for said drugs. The present invention also relates to a kit comprising the present human NSCLC cell lines. The present invention also disclosed methods for evaluating the efficacy of anti-NSCLC drugs by using the human NSCLC cell lines.

### PRIORITY CLAIMS

This application claims priority to International Application numbers PCT/CN/2021/098081 filed on Jun. 3, 2021 and PCT/CN/2020/094778 filed on Jun. 5, 2020.

### TECHNICAL FIELD

The present invention relates to a human non-small cell lung cancer (NSCLC) cell line, and the use of the same in evaluating the efficacy of anti-NSCLC drugs. The present invention also discloses an animal model comprising the human NSCLC cell lines, method for producing the same and the use of the animal model in testing efficacy of drugs or obtaining pharmacological data for said drugs. The present invention also relates to a kit comprising the present human NSCLC cell lines. The present invention also disclosed methods for evaluating the efficacy of anti-NSCLC drugs by using the human NSCLC cell lines.

### BACKGROUND

Lung cancer is one of the most common malignant tumors in the world, which has become the first-ranking cause of death of malignant tumors in urban population of China Non-small cell lung cancer (NSCLC) includes squamous cell carcinoma (SCC), adenocarcinoma and large cell carcinoma. Compared with small cell carcinoma, the growth and division of cancer cells are slower, and the diffusion and metastasis are relatively late. Non-small cell lung cancer accounts for about 80% of all lung cancer patients. About 75% of the patients have been found in the middle and late stage, and the 5-year survival rate is very low.

Cell line refers to the colony of cells which are propagated after the first passage of primary cell culture. It also refers to the culture cells that can be passed on continuously for a long time. However there are very few cells that can survive and continue to pass on, i.e. very few of them can become cell lines.

EGFR (epithelial growth factor receptor) is the receptor of EGF cell proliferation and signal transduction. Studies have shown that EGFR is highly or abnormally expressed in many solid tumors. EGFR is related to tumor cell proliferation, angiogenesis, tumor invasion, metastasis and inhibition of apoptosis. Mutations in the tyrosine kinase region of EGFR mainly occur in exons 18-21, and mutations in exons 19 and 21 covered 90% of all EGFR mutations. EGFR mutations can be detected by using e.g. PCR and direct sequencing techniques. EGFR plays an important role in the proliferation, growth, repair and survival of tumor cells. EGFR is overexpressed in many epithelial tumors, such as non-small cell lung cancer, breast cancer, glioma, head and neck cancer, cervical cancer, bladder cancer, gastric cancer, etc. In addition, the abnormal expression of EGFR is closely related to neovascularization, tumor invasion and metastasis, chemotherapy resistance and prognosis. Nearly 80-90% of the EGFR mutations are either small exon 19 deletions or the L858R mutation in exon 21, but other TKIs sensitive EGFR mutations can occur in exons 12, 19, 20, 21. Mutations associated with TKIs resistance, like the T790M in exon 20, can also develop in small tumor cell sub-clones and need to be identified (Dario de Biase et al., “Next-Generation Sequencing of Lung Cancer EGFR Exons 18-21 Allows Effective Molecular Diagnosis of Small Routine Samples (Cytology and Biopsy)”, published on Dec. 23, 2013, volume 8. Issue 12. e83607, https://doi.org/10.1371/journal.pone.0083607). Method for detecting EGFR mutations in NSCLC cells is known in the art. See e.g. Huili Chu et al, “Direct sequencing and amplification refractory mutation system for epidermal growth factor receptor mutations in patients with non-small cell lung cancer”, published online on: Aug. 29, 2013, https://doi.org/10.3892/or.2013 3709 pages: 2311-2315, and Ching-Hsiung Lin et al, “Rapid detection of epidermal growth factor receptor mutations with multiplex PCR and primer extension in lung cancer”, J Biomed Sci. 2010; 17(1): 37, Published online 2010 May 12. doi: 10.1186/1423-0127-17-37, http://www.jbiomedsci.com/content/17/1/37.

The present invention discloses novel NSCLC tumor cell lines with one or more EGFR mutations in Exons 19, 20 and 21 which have significant importance in studying the mechanism of cell carcinogenesis, tumor metastasis, as well as in evaluation drug efficacy for NSCLC. Establishing different tumor cell lines for cancer study can provide useful data for new drug research and development. Tumor cell lines with EGFR mutations are useful tools for evaluating anti-NSCLC drugs both in vitro and ex vivo. For example, a tumor cell line can be implanted subcutaneously to form an animal model for drug efficacy evaluation, which will mimic the in vivo environment of patients, reflect the patient's response and thus is more effective. Thus in the relevant technical field there is a need for NSCLC cell lines with one or more EGFR mutations in Exons 19, 20 and 21, as well as a wild-type NSCLC cell line of the same origin which can be used as a reference, wherein said NSCLC cell lines maintain stable genetic characteristics during proliferation and grow into tumor in an animal model. The human NSCLC cell lines of the present invention, the reagent, kit or animal model containing the same are proved to be very useful for drug research or development.

### SUMMARY OF THE DISCLOSURE

The present invention provides human NSCLC (hNSCLC) cell lines, i.e. LD1-0025-200636, LD1-0025-200694, LD1-0006-215676 and LD1-0025-200717.

In particular, the human NSCLC (hNSCLC) cell line LD1-0025-200636 is directly established from EGFR WT NSCLC patient; LD1-0025-200694 is directly established from EGFR L858R NSCLC patient; LD1-0006-215676 is directly established from EGFR double-mutant (L858R/T790M) patient; and LD1-0025-200717 is directly established from EGFR triple-mutant (19del/T790M/C797S) patient.

The human NSCLC cell lines of the present invention can proliferate in vitro and grow into tumors in animal models while maintaining their genetic characteristics.

The present invention also comprises the progeny cell lines of LD1-0025-200636, LD1-0025-200694, LD1-0006-215676 and LD1-0025-200717.

In embodiments, the present invention relates to a method for generating hNSCLC cell lines of the present invention from a NSCLC sample of a patient by serial passaging. In embodiments, the hNSCLC cell lines of the present invention have been passed for over 10 generations, more particularly over 20 generations, more particularly over 30 generations, more particularly over 50 generations, more particularly over 70 generations, more particularly over 100 generations. Preferably the hNSCLC cell lines of the present invention have been passed for 10 generations, 30 generations, 35 generations, 40 generations, 45 generations, 50 generations, 55 generations, 60 generations, 65 generations, 70 generations, 75 generations, 80 generations, 85 generations, 90 generations, 95 generations and 100 generations.

The present invention also relates to a sample of tissue or organ comprising the hNSCLC cell lines of the present invention. In particular, said sample of tissue or organ are derived from animal models implanted or injected with the hNSCLC cell lines of the present invention. In embodiments, said tissue or organ is derived from the lung of animal models, preferably immune compromised mice. In embodiments, said sample of tissue or organ comprises metastatic hNSCLC cell lines of the present invention, in particular said sample of tissue or organ comprises metastatic hNSCLC cell lines of the present invention in the brain or bone of a mammal.

In one aspect the present invention relates to a reagent comprising the hNSCLC cell lines of the present invention. Said reagent can be used to evaluating anti-hNSCLC drugs.

In another aspect, the present invention also relates to a kit comprising the hNSCLC cell lines of the present invention or a sample of tissue or organ comprising the hNSCLC cell lines of the present invention. In embodiments the kit can be used to evaluate the efficacy anti-NSCLC drugs or used in drug research and development to obtain pharmacological data or other relevant data. In embodiments, the kit of the present invention further comprises means or reagent for detecting the presence of the hNSCLC cell lines of the present invention.

In another aspect, the present invention discloses an animal model comprising the hNSCLC cell lines of the present invention. In embodiments, the animal model is obtained by implanting or injecting the hNSCLC cell lines of the present invention subcutaneously into the animal. In embodiments, said animal model is an immune compromised mammal, preferably an immune compromised mouse.

The present invention also discloses methods for evaluating drug efficacy using the hNSCLC cell lines of the present invention, the reagent or kit comprising the hNSCLC cell lines of the present invention. In embodiments, the hNSCLC cell lines of the present invention, the reagent comprising the bNSCLC cell lines of the present invention or the kit of the present invention are used to obtain data for drug research and development. In embodiments, the data obtained from the hNSCLC cell lines of the present invention, the reagent comprising the hNSCLC cell lines of the present invention or the kit of the present invention are used to set up a computer model for drug efficacy evaluation, in particular said data is processed by a computer software to obtain a computer model for evaluating the efficacy of anti-NSCLC drugs.

### Deposit Information of Biological Material

The present invention provides a human NSCLC cell line, LD1-0025-200636, deposited with China Center for Type Culture Collection (CCTCC) under CCTCC No. C202005 on Jun. 3, 2020. Address: Wuhan University, Wuhan, China, 430072.

The present invention provides a human NSCLC cell line, LD1-0025-200694, deposited with China Center for Type Culture Collection (CCTCC) under CCTCC No. C2020102 on Jun. 3, 2020. Address: Wuhan University, Wuhan, China, 430072.

Human NSCLC cell line of the present invention, LD1-0006-215676, deposited with China Center for Type Culture Collection (CCTCC) under CCTCC No. C2020104 on Jun. 3, 2020. Address: Wuhan University, Wuhan, China, 430072.

Human NSCLC cell line of the present invention, LD1-0025-200717, deposited with China Center for Type Culture Collection (CCTCC) under CCTCC No. C2020103 on Jun. 3, 2020. Address: Wuhan University, Wuhan, China, 430072.

### BRIEF DESCRIPTION OF THE DRAWINGS

FIG. 1 shows the results of cell proliferation test for LD1-0025-200636 (EGFR WT cell line, FIG. 1A) and LD1-0006-215676 (FIG. 1B) and LD1-0025-200717 (FIG. 1C).

FIG. 2A illustrates the EGFR mutations in Exons 20 and 21 encoding T790M and L858R detected in the hNSCLC cell line LD1-0006-215676: FIG. 2B illustrates the EGFR mutations in Exon 20 encoding T790M, C797S and the Exon 19 deletion 746_750del (“19 deletion”) detected in the hNSCLC cell line LD1-0025-200717.

FIG. 3A. Inhibition curve of LD1-0025-200636 (EGFR WT cell line). B. Inhibition curve of LD1-0025-200694 (EGFR L858R) cell line. C. Inhibition curve of LD1-0006-215676 cell line. D. Inhibition curve of LD1-0025-200717 cell line.

### DETAILED DESCRIPTION

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

The present invention provides a human NSCLC cell line, LD1-0025-200636, which is deposited with China Center for Type Culture Collection (CCTCC) under CCTCC No. C202005 on Jun. 3, 2020. Address: Wuhan University, Wuhan, China, 430072.

In embodiments, the present invention provides a human NSCLC cell line, LD1-0025-200694, which is deposited with China Center for Type Culture Collection (CCTCC) under CCTCC No. C2020102 on Jun. 3, 2020. Address: Wuhan University, Wuhan, China, 430072. Cell line LD1-0025-200694 comprises EGFR mutations in Exon 21 encoding L858R.

In embodiments, the present invention provides a human NSCLC cell line. LD1-0006-215676, which is deposited with China Center for Type Culture Collection (CCTCC) under CCTCC No. C2020104 on Jun. 3, 2020. Address: Wuhan University, Wuhan, China, 430072. Cell line LD1-0006-215676 comprises EGFR mutations in Exons 20 and 21 encoding T790M and L858R.

In embodiments, the present invention provides a human NSCLC cell lines, LD1-0025-200717, which is deposited with China Center for Type Culture Collection (CCTCC) under CCTCC No. C2020103 on Jun. 3, 2020. Address: Wuhan University, Wuhan, China, 430072. Cell line LD1-0025-200717 comprises the EGFR mutations in Exon 20 encoding T790M and C797S and the Exon 19 deletion 746_750del.

In particular, the human NSCLC (hNSCLC) cell line LD1-0025-200636 is directly established from EGFR WT NSCLC patient; LD1-0025-200694 is directly established from EGFR L858R NSCLC patient; LD1-0006-215676 is directly established from EGFR double-mutant (L858R/T790M) patient; and LD1-0025-200717 is directly established from EGFR triple-mutant (19del/T790M/C797S) patient.

Accordingly, in embodiments, the present invention provides a PDX (Patient-Derived Xenograft) model generated from NSCLC patients, including EGFR WT NSCLC patient, EGFR L858R. NSCLC patient, EGFR double-mutant (L858R/T790M) patient, and EGFR triple-mutant (19del/T790M/C797S) patient. Since a PDX model retains the characteristics of the primary patient tumor including gene expression profiles and drug responses, it is the most reliable in vivo human cancer model.

Treatment of non-small cell lung cancer (NSCLC) patients with activating EGFR mutations using epidermal growth factor receptor (EGFR) tyrosine kinase inhibitors (TKIs) represents & milestone achievement in the targeted therapy of NSCLC. However, unavoidable emergence of acquired resistance limits the long-term benefits of patients in the clinic. The appearance of EGFR C797S mutation caused by the third generation EGFR-TKI, osimertinib, as second-line treatment on top of T790 mutation is particularly a challenge, making the treatment failure eventually. Lack of naturally derived cell lines or patient-derived xenografts (PDXs) harboring triple mutations of EGFR at 19del, T790M and C797S substantially slows down our understanding on the biology of acquired resistance to osimertinib and development of effective strategies for overcoming acquired resistance caused by C797S mutation.

To this end, the present inventors have successfully established a PDX from a NSCLC patient harboring 19del and T790M mutated EGFR who became resistant to osimertinib due to appearance of C797S. Moreover, the inventors have generated a cell line from this EGFR triple mutated PDX. Hence, this PDX together with its matched cell line provides with valuable research models for understanding and overcoming acquired resistance to osimertinib due to EGFR. C797S mutation.

Availability of cell lines and patient-derived PDXs with C797S mutation is essential for developing effective agents or strategies to overcome osimertinib resistance caused by acquisition of C797S mutation. Unfortunately, these patient-derived cell lines and PDXs are lacking in our research community except for an engineered EGFR mutant cell lines expressing 19del, T790M and C797S triple mutated EGFR. Although in vivo model, cell-derived xenografts (CDX) may be established by engineered EGFR mutant cell lines, but CDX models are limited by their reduced intra-tumoural heterogeneity and their poor record of predicting clinically effective therapies (Whittle et al., 2015 and references therein). In addition, engineered triple mutated EGFR mutant cell lines could not fully mimic the complicated natural mechanism of resistance to osimertinib, since the drug resistance may be caused by multiple factors including C797S.

In embodiments, the present invention relates to the progeny cell lines derived from the hSCLC cell lines of the present invention.

In embodiments, the present invention relates to a method for generating bNSCLC cells lines of the present invention from a sample of patient by serial passaging. In embodiments, the hNSCLC cell lines of the present invention has been passed down for over 10 generations, more particularly over 20 generations, more particularly over 30 generations, more particularly over 50 generations, more particularly over 70 generations, more particularly over 100 generations. Preferably the hNSCLC cell lines of the present invention has been passed for 10 generations, 30 generations, 35 generations, 40 generations, 45 generations, 50 generations, 55 generations, 60 generations, 65 generations, 70 generations, 75 generations, 80 generations, 85 generations, 90 generations, 95 generations and 100 generations. In embodiments, the hNSCLC of the present invention is obtained by a method comprising the following steps: (a) implanting tumor tissue or injecting tumor cells obtained from the patient into an immune compromised animal, (b) dissecting the tissue or organ comprising the tumor cells from the immune compromised animal to collect the tumor cells; (c) removing away non-tumor tissue and necrotic tumor tissue and cutting the tumor into 1˜2 mm³ fragments before suspending pellet with digestion buffer and incubate at 37° C.; (d) collecting single cells through 70 uM strainer and centrifuged at 1,000 rpm for 3 min; (e) suspending the cells and centrifuged with 10 ml Histopaque solution and collecting cells from middle layer; (f) cells are cultured using Conditional Reprogramming Cell Culture Kit (available from Shanghai Lide), (g) passing cells for 10-100 generations; (h) harvesting the stable cell line. In embodiments, the sample of the patient derives from an in-situ NSCLC tissue or a metastatic NSCLC tissue.

The present invention also relates to a sample of tissue or organ comprising the hNSCLC cell lines of the present invention. In particular, said sample of tissue or organ are derived from mammals comprising the hNSCLC cell lines of the present invention. In embodiments, said tissue or organ is derived from the lung of immune compromised mammals, preferably immune compromised mice. In embodiments, said sample of tissue or organ comprises metastatic hNSCLC cell lines of the present invention, wherein said sample of tissue or organ are derived from the brain or bone of the animal.

In another aspect, the present invention relates to a reagent comprising the hNSCLC cell lines of the present invention or a sample of tissue or organ comprising the hNSCLC cell lines of the present invention. In embodiments the reagent can be used to evaluate anti-NSCLC drugs or used in drug research and development to obtain pharmacological data or other relevant data.

In another aspect, the present invention relates a kit comprising the hNSCLC cell lines of the present invention or a sample of tissue or organ comprising the hNSCLC cell lines of the present invention. In embodiments the kit can be used to evaluate anti-NSCLC drugs or used in drug research and development to obtain pharmacological data or other relevant data. In embodiments the kit further comprises reagents or means for detecting the presence of the human NSCLC cell lines of the present invention.

In another aspect, the present invention discloses an animal model comprising the hNSCLC cell lines of the present invention. In embodiments, the animal model is obtained by implanting or injecting the hNSCLC cell lines of the present invention subcutaneously into the animal. In embodiments, said animal is an immune comprised mammal. In embodiments, said immune comprised mammal is a mouse.

The present invention also discloses methods for evaluating drug efficacy using the hNSCLC cell lines of the present invention, the reagent, kit or animal model comprising the hNSCLC cell lines of the present invention. In embodiments, the hNSCLC cell lines of the present invention, the reagent, kit or animal model comprising the hNSCLC cell lines of the present invention are used to obtain data for drug research and development. In embodiments, the data obtained from the hNSCLC cell lines of the present invention, the reagent, kit or animal model comprising the hNSCLC cell lines of the present invention are used to set up a computer model for drug screening, in particular said data is processed by computer software to obtain a computer model for efficacy of anti-NSCLC drugs.

The anti-NSCLC drug to be evaluated can be administered via any suitable route, either oral or parental. For example, the candidate drug is administered to the animal model of the present invention by oral administration or intramuscular injection (such as through intramuscular, subcutaneous or intravenous infusion), local administration, inhalation, and transdermal delivery such as skin patches, implants, suppositories, etc. A skilled person in the art will choose a suitable route of administration according to their needs.

The anti-NSCLC drugs to be evaluated in the present invention may be a known antitumor drug or its combination, a new antitumor or a combination, or a new combination of known antitumor drugs. In the method of the invention, the drug to be screened can be used in solid, semi-solid or liquid form.

The animal model of the present invention can be administered with the anti-NSCLC drugs by oral administration or intramuscular injection (such as through intramuscular, subcutaneous or intravenous infusion), local administration, inhalation, and transdermal delivery such as skin patches, implants, suppositories, etc. Persons skilled in the art will choose a suitable route of administration according to their needs.

In embodiments, cell proliferation of hNSCLC cell lines of the present invention is tested by CTG (CELL TITER-GLO), Kits for use in the CTG tests are commercially available. In embodiments, genomic profiling for the hNSCLC cell lines of the present invention is tested by Sanger Sequencing, a method which is known to the skilled person in the art.

The present invention also provides a method or use of the hNSCLC cell lines of the present invention, the reagent, kit or animal model comprising the hNSCLC cell lines of the present invention in obtaining drug efficacy data for generating a drug efficacy database for hNSCLC cells containing the EFGR mutations in the Exons 19-21. In embodiments the drug efficacy database include inhibition rate of cell growth of one or more candidate drugs. In embodiments, the present invention provides a computer readable medium for implementing for following steps:
(i) calculating the percentage of inhibition of the growth of the hNSCLC cell lines of the present invention after contacted with one or more candidate drugs according to the following formula:

Inhibition(%)=((Vcontrol group-Vvehicle group)−(Vdrug treatment group-Vvehicle group))/(Vcontrol group-Vvehicle group)*100%,
(ii) making an inhibition curve by XLfit(IDBS) and calculating the corresponding IC₅₀,
(iii) optionally building a computer accessible database including the name of said one or more candidate drugs, the percentage of inhibition of the growth of the hNSCLC cell lines of the present invention, the inhibition curve, and the corresponding IC₅₀.

### EXAMPLES

### Example 1. PDX Model Construction

LD1-0025-200636 PDX model was established from an EGFR WT NSCLC patient.
LD1-0025-200694 PDX model was established from an EGFR L858R NSCLC patient.
LD1-0006-215676 PDX model was established from pleural effusion sample obtained from EGFR double-mutant (L858R/T790M) patient.

LD1-0025-200717 PDX model was established from biopsy sample obtained from patient who received a diagnosis of NSCLC and became resistant to Osimertinib(AZD9291). The patient is EGFR triple-mutant (19del/T790M/C797S). The detailed history of treatment for the patient was showed as follows.

The EGFR mutations was progressively developed from 19del to 19del/T790M, then 19del/T790M/C797S. The influence of the treatment history could not be mimicked by any other artificial models. The naturally derived PDXs and cell lines will be valuable research tools or models for developing efficacious agents or strategies to combat acquired resistance to osimertinib.

TABLE 1
The WES (Whole Exome Sequencing) results of PDX model for the cell lines are
as follows. All mutations are retained in the corresponding PDX model.

| | Gene | | | | | | allele_freq | AA |
|---|---|---|---|---|---|---|---|---|
| Model ID | symbol | Function class | Chrom | Pos | ref | alt | (%) | change |
| LD1-0025-200636 | EGFR | WT | — | — | — | — | — | — |
| LD1-0025-200694 | EGFR | nonsynonymous | chr7 | 55259515 | T | G | 60.30534 | p.L858R |
| | | SNV | | | | | | |
| LD1-0006-215676 | EGFR | nonsynonymous | chr7 | 55249071 | C | T | 69.61538 | p.T790M |
| | | SNV | | | | | | |
| | EGFR | nonsynonymous | chr7 | 55259515 | T | G | 76.64336 | p.L858R |
| | | SNV | | | | | | |
| LD1-0025-200717 | EGFR | nonframeshift | chr7 | 55242465 | GGA | G | 45.9016 | Exon19, |
| | | substitution | | | ATT | | | 746_750del |
| | | | | | AAG | | | |
| | | | | | AGA | | | |
| | | | | | AGC | | | |
| | | | | | A | | | |
| | EGPR | nonsynonymous | chr7 | 55249071 | C | T | 17.3745 | T790M |
| | | SNV | | | | | | |
| | EGFR | nonsynonymous | chr7 | 55249092 | G | C | 16.726 | C797S |
| | | SNV | | | | | | |

### Example 2. Cell Line Establishment

PDX tumors were collected and immersed in HBSS: Washed the tumor tissue with HBSS and removed away non-tumor tissue and necrotic tumor tissue in biosafety cabinet. Cut the tumor into 1˜2 mm³ fragments and suspended pellet with digestion buffer and incubate in 37° C. for. 2:4 hours. Collected single cells through 70 uM strainer and centrifuged at 1,000 rom for 3 min. Suspended the cells and centrifuged with 10 ml Histopaque solution and collected cells from middle layer. Suspended cells and cultured cells using Conditional Reprogramming Cell Culture Kit. Serially passing cells for more than 10 generations until the stable cell line is obtained.

### Example 3. Cell Proliferation and Genomic Profiling

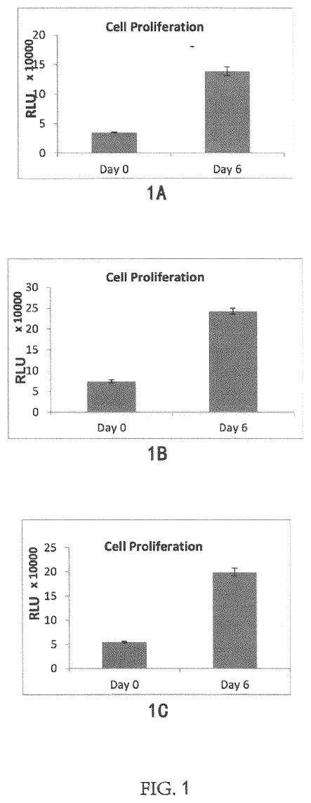

Cell proliferation was test by CTG. Genomic profiling was test by Sanger Sequencing.
LD1-0025-200636: The cell generated 4.0-fold proliferation with good cell viability. (FIG. 1A)
LD1-0006-215676: The cell generated 3.3-fold proliferation with good cell viability. (FIG. 1B)
LD1-0025-200717: The cell generated 3.7-fold proliferation with good cell viability. (FIG. 1C)

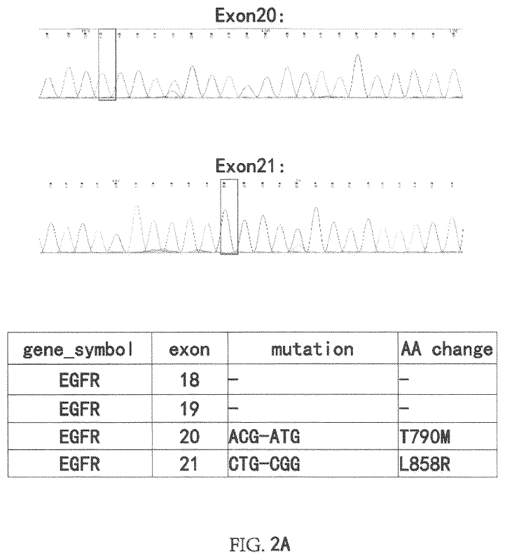

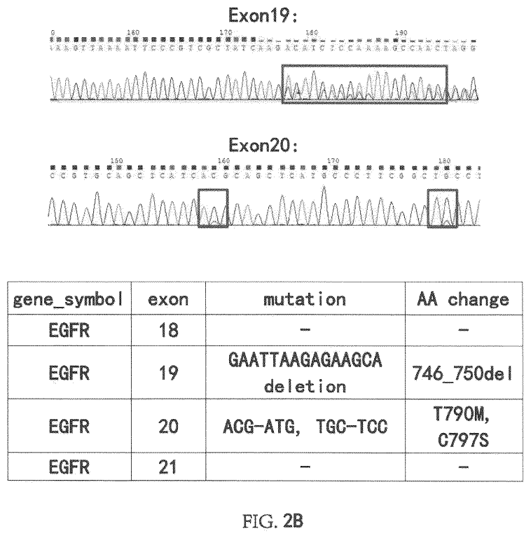

The results of genome profiling for the cell lines of the present invention are shown in FIG. 2.

The EGFR mutations in the hNSCLC cell lines of the present invention were confirmed by Sanger sequencing. After passing for 9-50 generations, detecting the mutations in the hNSCLC cell lines of the present invention by Sanger sequencing again to identify all mutations remained in the relevant cell lines.

### Example 4

This study was conducted to investigate the authenticity of the above newly established cell lines using short tandem repeat (STR) DNA profiling method. The protocol is as follows:
1. Genomic DNA was extracted from the cell pellet and PDX tissue
2. Samples, together with positive and negative control were amplified using GenePrint 10 System (Promega).
3. Amplified products were processed using the ABI3730xlGenetic Analyzer.
4. Data were analyzed using GeneMapper4.0 software and then compared the cell with the matching PDX tissue.

The details of the cell lines for STR profiling; and the results are shown in Tables 2-3 below.

TABLE 2
Details of the cell lines for STR profiling.

| External ID | Gene symbol | Mutation |
|---|---|---|
| LD1-0025-200636 | EGFR | WT |
| LD1-0025-200694 | EGFR | L858R |
| LD1-0006-215676 | EGFR | L858R/T790M |
| LD1-0025-200717 | EGFR | 19del/T790M/C797S |

TABLE 3
Percent match between the primary cell line and the PDX profile

| Genetic Site | Primary Cell line information | | PDX information | |
|---|---|---|---|---|
| (Locus) | LD1-0025-200636 cell | | LD1-0025-200636 PDX | |
| Amelogenin | X | Y | X | Y |
| CSF1PO | 11 | 11 | 11 | 11 |
| D13S317 | 8 | 9 | 8 | 9 |
| D168539 | 13 | 13 | 13 | 13 |
| DSS818 | 11 | 11 | 11 | 11 |
| D75820 | 11 | 11 | 11 | 11 |
| THO1 | 7 | 9 | 7 | 9 |
| TPOX | 8 | 9 | 8 | 9 |
| vWA | 16 | 18 | 16 | 18 |
| D21S11 | 29 | 31 | 29 | 31 |
| Genetic Site | Primary Cell line information | | PDX information | |
| (Locus) | LD1-0025-200694 cell | | LD1-0025-200694 PDX | |
| Amelogenin | X | X | X | X |
| CSF1PO | 11 | 11 | 11 | 11 |
| D13S317 | 8 | 11 | 8 | 11 |
| D16S539 | 12 | 12 | 12 | 12 |
| D5S818 | 10 | 10 | 10 | 10 |
| D7S820 | 12 | 12 | 12 | 12 |
| THO1 | 9 | 9 | 9 | 9 |
| TPOX | 11 | 11 | 11 | 11 |
| vWA | 17 | 17 | 17 | 17 |
| D21S11 | 29 | 31 | 29 | 31 |
| Genetic Site | Primary Cell line information | | PDX information | |
| (Locus) | LD1-0006-215676 cell | | LD1-0006-215676 PDX | |
| Amelogenin | X | X | X | X |
| CSF1PO | 12 | 12 | 12 | 12 |
| D13S317 | 10 | 10 | 10 | 10 |
| D16S539 | 11 | 11 | 11 | 11 |
| D5S818 | 13 | 13 | 13 | 13 |
| D7S820 | 11 | 11 | 11 | 11 |
| THO1 | 9 | 10 | 9 | 10 |
| TPOX | 8 | 8 | 8 | 8 |
| vWA | 14 | 14 | 14 | 14 |
| D21S11 | 30 | 31 | 30 | 31 |
| Genetic Site | Primary Cell line information | | PDX information | |
| (Locus) | LD1-0025-200717 cell | | LD1-0025-200717 PDX | |
| Amelogenin | X | X | X | X |
| CSF1PO | 12 | 12 | 12 | 12 |
| D13S317 | 12 | 12 | 12 | 12 |
| D16S539 | 9 | 11 | 9 | 11 |
| D5S818 | 9 | 10 | 9 | 10 |
| D7S820 | 11 | 12 | 11 | 12 |
| THO1 | 7 | 9 | 7 | 9 |
| TPOX | 8 | 8 | 8 | 8 |
| vWA | 14 | 15 | 14 | 15 |
| D21S11 | 29 | 30 | 29 | 30 |

Percent match between the primary Cell line and the PDX profile: 100%

This Example confirms the authenticity of the newly established cell lines.

### Example 5. Drug Efficacy Study

The cell lines of the present disclosure (i.e., LD1-0025-200636; LD1-0025-200694; LD1-0006-215676; and LD1-0025-200717) were seeded at 2×10² cells/well to 96 well round bottom ultra low attachment plate. The drugs were added into the cell lines and the cells were cultured in 37° C. and 5% CO₂ for 6 days. The ATP amount was measured using CellTiter-Glo. The Inhibition rate corresponding to the concentration of the drugs was calculated. The equation for calculating the inhibition rate is as follows:

$$Inhibition ( \% ) = ( ( {V}_{emitted group} \cdot {V}_{vehicle group} ) - ( V \text{?} - {V}_{vehicle group} ) ) / {V}_{control group} - {V}_{vehicle group} ) * 100 \% .$$

$$\text{?} \text{indicates text missing or illegible when filed}$$

Inhibition curve was graphed by XLfit(IDBS) and IC50 was calculated.

In vitro efficacy study of AZD9291 for the NSCLC cell lines

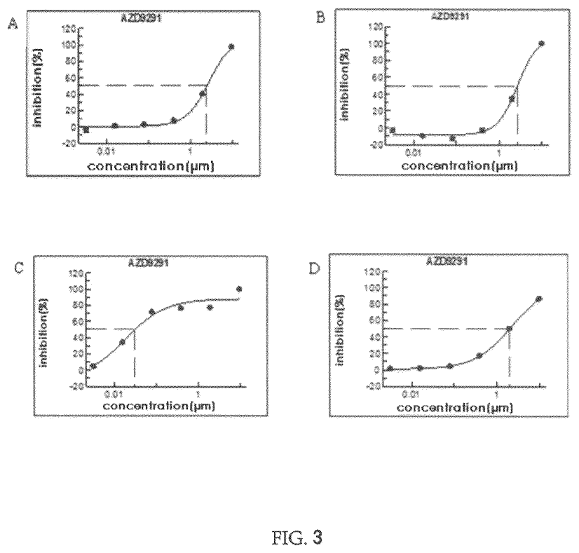

The in vitro efficacy study of AZD9291 for the NSCLC cell lines of the present disclosure was performed (i.e., LD1-0025-200636; LD1-0025-200694; LD1-0006-215676; and LD1-0025-200717). Results were shown in FIG. 3:A. Inhibition curve of LD1-0025-200636 (EGFR WT) cell line. B. Inhibition curve of LD1-0025-200694 (EGFR L858R) cell line. C. Inhibition curve of LD1-0006-215676 (EGFR L858R/T790M) cell line. D. Inhibition curve of LD1-0025-200717 (EGFR 19del, T790M & C797S mutant) cell line.

TABLE 4
IC₅₀ of AZD9291 in four hNSCLC cell lines. Mean Inhibition
shown for each dose (triplicate). Error bars, SEM.

| Model ID | EGFR status | IC 50 (μm) |
|---|---|---|
| LD1-0025-200636 | WT | 2.52 |
| LD1-0025-200694 | L858R | 2.67 |
| LD1-0006-215676 | L858R/T790M | 0.03 |
| LD1-0025-200717 | 9del/T790M/C797S | 2.01 |

Within PDX models, tumor cells grow in physiologically-relevant tumor microenvironments that mimic the oxygen, nutrient, and hormone levels that are found in the patient's primary tumor site. Furthermore, implanted tumor tissue maintains the genetic and epigenetic abnormalities found in the patient. This study has found that PDX models exhibit similar responses to anti-cancer agents (i.e., AZD9291) as seen in the actual patient who provided the tumor sample. Hence, this Example demonstrated that PDX model is beneficial to test therapeutic responses for NSCLC drugs.

### Example 6. CDX (Cell-Line-Derived Xenograft) Animal Model Construction

NCG Mice are Triple Immunodeficient and Lack Functional/Mature T, B, and NK Cells, and have reduced macrophage and dendritic cell function. These animal models have the ability to host xenograft cells, tissue, and human immune system components. The characteristics of NCG mouse models make them ideal for oncology research.

The cells from NSCLC cell line of the present disclosure (i.e., LD1-0025-200636; or LD1-0025-200717) were digested to single cell by trypsin. The cells were counted and 5×10⁶ cells were transferred to the centrifuge rube. The cells were washed with PBS, centrifuged and the supernatant was removed away. The pellet was suspended with 100 uL PBS and the tube was placed on ice at once. The cell suspension was mixed with 100 uL. Matrigel on ice and the NOG mice were inoculated with the mixture subcutaneously. After cells transplantation, the animals are checked daily for morbidity and tumor development. It was observed that the CDX models for NSCLC cell lines of the present disclosure (i.e., LD1-0025-200636 and LD1-0025-200717) were successfully established.

The generation of CDX and PDX models in the present disclosure has suggested that the cell lines of the present disclosure can be used as a promising tool for preclinical drug development.

It is understood that the examples and embodiments described herein are for illustrative purposes only and that various modifications or changes in light thereof will be suggested to persons skilled in the art and are to be included within the spirit and purview of this application and scope of the appended claims.

I claim:

1-13. (canceled)

14. Human non-small cell lung carcinoma cell line LD1-0025-200717, deposited under CCTCC No. C2020103.

15. The cell line of claim 14, which comprises the EGFR mutations in Exon 20 encoding T790M and C797S as well as the Exon 19 deletion 746_750 del.

16. A method for evaluating efficacy of chemotherapeutic drugs, comprising,
contacting one or more chemotherapeutic drugs with the cell line of claim 14, and
determining inhibition of growth of the cell line.

17. A method for producing an animal model, comprising implanting the cell line of claim 14 into an animal, wherein the animal is an immune compromised mouse.

18. A kit comprising the cell line of claim 14.

19. Human non-small cell lung carcinoma cell line LD1-0025-200636, deposited under CCTCC No. C202005.

20. A method for evaluating efficacy of chemotherapeutic drugs, comprising,
contacting one or more chemotherapeutic drugs with the cell line of claim 19, and
determining inhibition of growth of the cell line.

21. A method for producing an animal model, comprising implanting the cell line of claim 19 into an animal, wherein the animal is an immune compromised mouse.

22. A kit comprising the cell line of claim 19.

23. Human non-small cell lung carcinoma cell line LD1-0025-200694, deposited under CCTCC No. C2020102.

24. The cell line of claim 23, which comprises EGFR mutations in Exon 21 encoding L858R.

25. A method for evaluating efficacy of chemotherapeutic drugs, comprising,
contacting one or more chemotherapeutic drugs with the cell line of claim 23, and
determining inhibition of growth of the cell line.

26. A kit comprising the cell line of claim 23.

27. Human non-small cell lung carcinoma cell line LD1-0006-215676, deposited under CCTCC No. C2020104.

28. The cell line of claim 27, which comprises EGFR mutations in Exons 20 and 21 encoding T790M and L858R.

29. A method for evaluating efficacy of chemotherapeutic drugs, comprising,
contacting one or more chemotherapeutic drugs with the cell line of claim 27, and
determining inhibition of growth of the cell line.

30. A method for producing an animal model, comprising implanting the cell line of claim 24 into an animal, wherein the animal is an immune compromised mouse.

31. A kit comprising the cell line of claim 23.
